(19) United States
(12) Patent Application Publication
(10) Pub. No.: US 2022/0137018 A1
(43) Pub. Date: May 5, 2022

(54) METHOD FOR IDENTIFYING AN ITEM BY OLFACTORY SIGNATURE

(71) Applicant: ARYBALLE, GRENOBLE (FR)

(72) Inventors: Samuel GUILAUMÉ, SAINT MARTIN D'URIAGE (FR); David SLOO, PARIS (FR)

(73) Assignee: ARYBALLE, GRENOBLE (FR)

(21) Appl. No.: 17/431,526

(22) PCT Filed: Feb. 18, 2020
(86) PCT No.: PCT/FR2020/050294

§ 371 Date: Aug. 17, 2021

(30) Foreign Application Priority Data
Feb. 18, 2019 (FR) 19 01602

(51) Int. Cl. G01N 33/00; G06K 9/62

(52) U.S. Cl. CPC G01N 33/0034; G01N 33/0036; G01N 33/025; G06K 9/6215; G06K 9/6276; G06K 9/6248

(57) ABSTRACT

A method implemented by a computer processing circuit connected to an electronic nose, for identifying a given item by an olfactory signature, the method making use of the electronic nose to obtain an olfactory signature, repeating the use of the electronic nose a first number K of times in order to acquire K olfactory signatures, making use of the computer processing circuit in order to estimate, on the basis of the K olfactory signatures, a model of the olfactory signature of the given item, acquiring, with an electronic nose of the same type, a current measurement of the olfactory signature of a current item of the same type as the given item, and comparing the current measurement to the model, in order to estimate a similarity (SIM) between the current item and the given item.

### TECHNICAL FIELD

The invention relates to a method for identifying an item by olfactory signature. In particular, the invention relates to a method for measuring a change in olfactory signatures over time. The invention also relates to a device configured to implement such a method.

The traceability of items, particularly in order to monitor the transport conditions of goods intended for mass distribution, today constitutes a major issue in supply chains. During the different phases in the supplying of items from a given producer to consumers, for example during storage and transport stages (by sea, road, etc.), these goods can undergo transformations affecting their value. Thus, impacts that occur during the transport of harvested fruit, or significant variations in the temperature of perfume bottles stored in a container, can be harmful to their initial quality.

Often, at the end of the supply chain, items are observed to be damaged and cannot be distributed. Too many transactions, rough transport, or even poorly packed storage are all events involving transformations that adversely affect the value of these items, and therefore the reliability of their supply chains. This is particularly true in the case of perishable items, their ripeness and freshness being directly affected by such events.

For tracking items over the course of a supply chain, methods and devices are known for measuring and recording their condition and position at a given time. The collection of these data allows various stakeholders to verify the reliability of the supply chain for these items. For example, there are NFC chips (“Near Field Communication”) or RFID chips (“Radio Frequency Identification”) to identify an item at a given moment and to track its position over time.

A disadvantage of known methods and devices is that they do not allow establishing an accurate history of the supply chain. At the end of the supply chain, and before delivery of items to a final recipient, it is therefore difficult to determine precisely when, where, and under what conditions items may have been damaged, on the basis of the data collected.

Another disadvantage of known methods and devices is that they do not guarantee that the measured data have not been altered during the supply chain, either intentionally or unintentionally. Although a comparison of data from different sensors, for example impact sensors placed on a container of bottles and position sensors, in principle allows deducing at what point in the supply chain these bottles may have been damaged, a third party can delete or falsify these data. The accessibility and precision of the data collected therefore do not always allow comparing them with each other. The origin and authenticity of these items are therefore not always verifiable.

Yet another disadvantage of known methods and devices is that they do not allow the condition of a perishable item to be precisely measured and tracked over time. However, the ripening of a perishable item, for example a fruit or a vegetable, is likely to vary more or less quickly depending on its environmental conditions, which the current sensors cannot reliably measure. Although it is possible to measure the temperature or humidity inside a container of food products, these measurements do not provide information about the ripening of these food products which depends expressly and in a complex manner on the environmental conditions, among many other factors related in particular to the type of perishable good.

There is therefore a need to provide a method improving the situation and not having at least one of the above disadvantages.

### OBJECT AND SUMMARY OF THE INVENTION

In order to respond to this or these disadvantages, the invention relates, according to a first aspect, to a method implemented by a computer processing circuit, connected to an electronic nose, for identifying a given item by an olfactory signature of the given item, said method comprising:
making use of the electronic nose comprising a plurality of sensors for detecting the presence of fluids likely to be present in a mixture of fluids originating from the given item, in order to obtain an olfactory signature of said mixture, the olfactory signature comprising respective proportions of said fluids in the mixture;
repeating the use of the electronic nose a first number K of times in order to acquire K olfactory signatures;
making use of the computer processing circuit to estimate, on the basis of said K olfactory signatures, a model of the olfactory signature of the given item;
acquiring, with an electronic nose of the same type, a current measurement of the olfactory signature of a current item of the same type as said given item; and
comparing the current measurement to said model, in order to estimate a similarity between the current item and the given item.

Herein, a given item comprises and releases a mixture of fluids. In particular, it releases part of its molecular material in the form of fluids such as gases or liquids, which can interact with sensors of an electronic nose such as the electronic nose described below, and comprising sensors such as odor or taste sensors or detectors of particular molecules in a gas or a liquid.

Herein, the computer processing circuit is any type of integrated circuit, this integrated circuit comprising a storage space, for example a memory, and a processor. The storage space is, for example, non-volatile memory (ROM or Flash, for example) and can constitute a storage medium, this storage medium also being able to comprise a computer program. The processor is a data processor which makes it possible to implement the instructions of a computer program. These instructions can be stored in a memory of a computing device, for example a server, then loaded and executed by the processor.

Herein, the first number K is an integer strictly greater than 1.

This makes it possible to detect a deterioration of the sensors of an electronic nose which could affect the accuracy of the olfactory signatures measured by said electronic nose. In practice, the sensors of an electronic nose comprise detection layers having a limited lifespan and such that after a certain number of measurements, these measurements are skewed and may no longer be reproducible. The aging of the sensors of an electronic nose can thus be detected and indicate the need for repair or replacement. This also makes it possible to provide a method for calibrating such an electronic nose, for example on the basis of olfactory signatures of samples of non-perishable items such as salt, sugar, water, or alcohol.

In one embodiment of the invention, the given item is a perishable item.

Herein, a perishable item is in general any item capable of transformation and not only towards expiration. A perishable item can be any type of item that cannot be stored for a long time under normal circumstances without alteration. A perishable item can also be a food product selected among any type of fruit, meat, fish, vegetables, dairy products, eggs, flour, cereals, and/or legumes. Similarly, a perishable item can be a gas, a liquid, or even a solid not intended for dietary consumption and whose physicochemical properties are liable to vary during different stages of a supply chain. A perishable item can also be perfume, gasoline, glue, motor oil, etc.

This makes it possible to detect changes in an olfactory signature of a given perishable item in order to identify a specific alteration, for example by comparing two olfactory signatures where a difference can indicate a different geographical origin or a counterfeit.

In one embodiment of the invention, the use of the electronic nose to obtain the first number K of olfactory signatures over time is carried out successively over time in order to acquire a succession of K olfactory signatures over time.

This makes it possible to build a model of the evolution over time based on several different measurements of the olfactory signatures of a mixture of fluids originating from a perishable item. It is thus possible to detect differences representative of a transformation of the components of this perishable item.

In one embodiment of the invention, the perishable item has several successive maturation phases over time and the K olfactory signatures are representative of said successive phases of the perishable item.

Herein, a maturation phase of a perishable item is any phase during which this item sees its physicochemical qualities change in a natural way, for example by aging, or in an unnatural way, for example after exposure to bacteria. In the case of perishable food products such as fruits, it is called physiological maturity when the fruit reaches its final stage of growth. This growth can be followed by a ripening process bringing it to a level of maturity that meets the standards required for distribution to a consumer; this level of maturity can be quantified using various physicochemical measurements, for example.

This makes it possible to quantify the maturation of a perishable item over time and to provide a reference from which several parameters of the item can be identified, for example its origin or abnormal degradations which have appeared during a supply chain for this item.

In one embodiment of the invention, the computer processing circuit is used to estimate, on the basis of said K successive olfactory signatures, a model of the evolution over time of the olfactory signature of the perishable item according to its various described states or maturation phase.

This makes it possible to construct models of the evolution of the maturation of a given perishable item based on estimated similarities between two items, or on the same item at successive moments. It is thus possible to precisely distinguish different perishable items according to their type, their geographical origin, differences in their rate of maturation, etc.

In one embodiment of the invention, the comparison of the current measurement to the model of the evolution over time gives an estimate of similarity between the current item and the perishable item at a given maturation phase of the perishable item.

This makes it possible to compare an olfactory signature with the model of the evolution after the fact, in order to deduce a deviation at a given moment from the maturation phase expected at the stage of the measurement.

In one embodiment of the invention, the model of the olfactory signature of the given item is obtained by multivariate analysis of the K olfactory signatures, each determined by the respective proportions of said fluids in the mixture, the model being defined in an L-dimensional space, the multivariate analysis being selected among principal component analysis or multidimensional scaling analysis.

Herein, the application of multivariate analysis makes it possible to convert respective measured proportions of fluids in a mixture which are correlated with each other into new variables which are uncorrelated with each other. The number L of dimensions is less than or equal to the first number K.

Alternatively, the model can be defined through other types of analysis such as through the use of neural networks, decision trees, random forests, etc.

In one embodiment of the invention, the multivariate analysis is selected among principal component analysis or multidimensional scaling analysis.

Herein, principal component analysis makes it possible to distinguish the most significant contributions among the respective proportions of fluids in a mixture, by reducing the number of variables used. This makes the information provided by them less redundant, while allowing a synthesis of relevant information in the form of principal components, which can be defined as “odors”, and resulting from a combination of the measured proportions.

Multidimensional scaling makes it possible to separate the respective proportions of the fluids in a mixture according to their variances, which also makes it possible to maximize the variance in an L-dimensional space. The distances are thus contrasted and the weakest contributions, usually due to noise measured by the sensors, can be eliminated more easily.

In one embodiment of the invention, the model is defined by a set of phase vectors in L-dimensional space, each phase vector characterizing a maturation phase of the perishable item.

Herein, a phase vector is a vector with L components that is determined as being a representative signature, for example a mean signature or a median signature, of a plurality of acquired olfactory signatures, this vector being characterized by a norm and a direction in L-dimensional space.

This makes it possible to quantify and represent a given maturation phase of a perishable item, based on a plurality of olfactory signatures selected to characterize said maturation phase.

In one embodiment of the invention, a distance is estimated, in the L-dimensional space, between a point representing the current measurement and each phase vector, the smallest of the estimated distances characterizing a current state of maturation of the perishable item.

This makes it possible to determine which maturation phase a current item is closest to, on the basis of a single current measurement.

In one embodiment of the invention, the distance is an absolute value, a Euclidean distance, or a distance between the point representing the current measurement and several nearest-neighbor phase vectors.

This makes it possible to make more precise the determination of a maturation phase to which a current item is closest, and in particular to better discriminate between several close maturation phases.

In one embodiment of the invention, the number of sensors comprised in the electronic nose is less than or equal to 100, and preferably equal to 25.

This makes it possible to ensure the acquisition of an optimal number of fluids present in a mixture of fluids originating from given items, in order to estimate a sufficiently precise model of the olfactory signature of these items while minimizing the size of the electronic nose.

In one embodiment of the invention, the method further comprises:
grouping several olfactory signatures into at least two groups, each group being defined by a center of mass of the signatures in said group, the distance of each signature from said center of mass being less than or equal to a predetermined distance;
comparing the distances between the current measurement and each of said groups, in order to associate the current item with the group to which the current item is closest.

Herein, the center of mass of a group of points in a 1-dimensional space corresponds to the median or to the mean of these points. In an L-dimensional space where L is greater than 1, the center of mass of a group of points, each point being defined by an L-tuple of coordinates, is a point for which each coordinate corresponds to the median or to the mean of the coordinates of these points which are of the same rank.

This makes it possible to identify and distinguish the olfactory signatures corresponding to different maturation phases according to different groups, to represent each group by a single virtual olfactory signature, called the center of mass, and to identify to which maturation phase a current item is closest.

Any other means of classification can be used indiscriminately to take into account the phase of a current item vector among the phases learned.

In one embodiment of the invention, the method further comprises:
making use of an additional sensor selected among a label sensor, a geolocation sensor, a temperature sensor, a pressure sensor, a humidity sensor, or an acceleration sensor, to measure a supplemental parameter of the given item;
acquiring, with an additional sensor of the same type, a current additional parameter of the current item, of the same type as the given item; and
associating said additional parameter with the group to which the current item is closest.

This makes it possible to improve the identification of the current item in relation to a given maturation phase based on measurements carried out by sensors which are not olfactory sensors, by matching a tag, a spatiotemporal value, a temperature value, a pressure value, a humidity value, or an acceleration value with this maturation phase.

In one embodiment of the invention, the method further comprises a processing of result data from said estimate of similarity between the current item and the given item, in order to protect said data against falsification.

Herein, and in a non-limiting manner, an exemplary data processing system implements a transmission-encoding device comprising means for encoding an olfactory signature and/or a result of an estimate of a similarity between a current item and a given item. This transmission-encoding device is for example comprised in the electronic nose or in the computer processing circuit, which further comprises means for transmitting the encoded information. This example of a processing system further comprises a reception-decoding device comprising means for receiving this encoded information and means for decoding it. This reception-decoding device is for example comprised in a human-machine or machine-machine interface.

This prevents a third party from altering the olfactory signatures acquired by the sensors of the electronic nose, as well as the result of an estimate of similarity between a current item and the given item.

In one embodiment of the invention, said result data are processed by a blockchain.

Herein, a blockchain is any type of distributed computing environment such as a networked client-server system with a user interface, in particular in combination with a data processing system.

This allows information to be stored and transmitted with enhanced security, through a consensus process within a network formed by several stakeholders in a supply chain. The use of a blockchain also preserves the reliability of the olfactory signature measurements carried out during a supply chain, which makes it possible to prevent further possible attempts at tampering. The processing of an olfactory signature and/or of result data within a blockchain provides a reliable guarantee of the measurements carried out within the framework of a given supply chain.

According to another aspect, the invention relates to a device for identifying a given item by its olfactory signature, the device comprising: —an electronic nose, comprising a plurality of sensors for detecting the presence of fluids likely to be present in a mixture of fluids originating from the given item, —a computer processing circuit, connected to the electronic nose in order to obtain an olfactory signature of said mixture, the olfactory signature comprising respective proportions of said fluids in the mixture, and the computer processing circuit being configured to implement the method according to one of the preceding claims.

According to another aspect, the invention relates to a computer program comprising instructions for all or part of a method as defined herein when said instructions are executed by a processor of a processing circuit.

### BRIEF DESCRIPTION OF DRAWINGS

Other features, details, and advantages of the invention will become apparent from reading the detailed description below, and from analyzing the accompanying drawings, in which:

FIG. 1, shows a view of an electronic nose for implementing a method according to the invention;

FIG. 2, shows a view of sensors of an electronic nose for implementing a method according to the invention;

FIG. 3, shows an exemplary representation of an olfactory signature acquired by an electronic nose;

FIG. 4, shows a schematic view of a method according to an exemplary implementation of the invention;

FIG. 5, shows an example of a model of the evolution over time of an olfactory signature of a perishable item;

FIG. 6, shows an exemplary implementation of a method according to the invention in the context of a supply chain;

FIG. 7, shows an example of a model defined by applying multivariate analysis to several olfactory signatures in order to define a model in a 2-dimensional space; and

FIG. 8, shows an example of a comparison between a current item and given items, by means of a set of phase vectors in a 2-dimensional space.

Unless otherwise indicated, similar or common elements in multiple figures bear the same reference symbols and have identical or similar characteristics, so these common elements are generally not described again for the sake of simplicity.

### DESCRIPTION OF EMBODIMENTS

Herein, an electronic nose is a physical device configured for acquiring an olfactory signature of a given object, such as a perishable item, from odors released by that item. An electronic nose typically comprises a plurality of sensors configured to recognize the presence of a target compound, for example a chemical or biological analyte, in a fluid such as a gas sample or liquid sample.

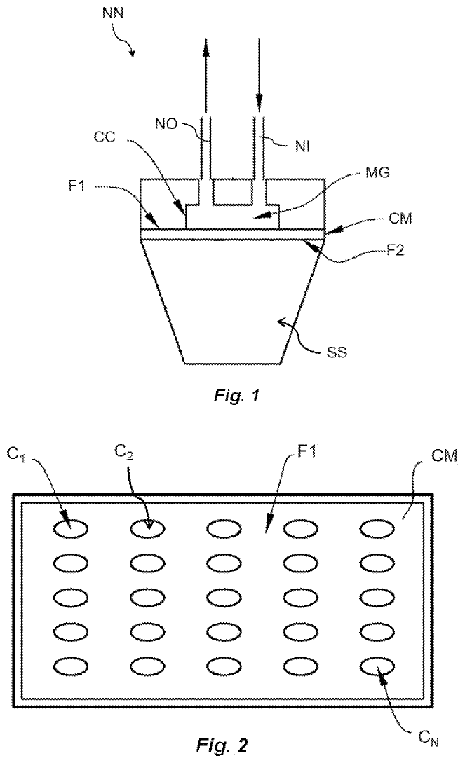

FIG. 1 shows an example of an electronic nose NN configured for implementing a method according to the invention. FIG. 2 is a view of a metal layer CM comprised in the electronic nose NN and its sensors C₁, C₂, . . . , C_{N}, this metal layer being provided to facilitate detection, and in particular adsorption, of fluids in a mixture in contact with the layer CM.

In the example presented here, the electronic nose NN comprises a metal layer CM, preferably flat, and comprising for example gold. The layer CM of the electronic nose NN further comprises a number N of sensors C₁, C₂, . . . , C_{N} formed on a first face F1 of the metal layer CM so that the first face F1 of the metal layer CM and said sensors are in contact with a mixture of a fluid, in particular a fluid of a dielectric nature, for example a liquid or a gas which is released by an item to be analyzed by means of the electronic nose NN.

In the example presented here, the number N of sensors comprised in the nose NN can vary between 1 and several hundred, preferably between 20 and 100. In a non-limiting manner, the examples presented here relate to a nose NN comprising 10 or 25 sensors in order to optimize the size of the nose NN while allowing it to maximize its sensitivity. Herein, the plurality of sensors comprises at least two sensors of different sensitivity in order to obtain an olfactory signature of said mixture.

In the example presented here, the electronic nose NN also comprises a support SS for said metal layer CM. The support SS is arranged against a second face F2 of the metal layer CM, this second face F2 being opposite to the first face F1. Generally, the support SS is formed from a dielectric material and has a refractive index greater than the refractive index of the mixture to be analyzed. This support SS is for example a glass prism.

In the example presented here, another metal layer (not shown) that is thin, for example made of Chromium (Cr), is provided between the second face F2 and the support SS, to ensure stable adhesion of the metal layer CM on the support SS.

In the example presented here, the electronic nose NN further comprises a suction system for capturing a volume sample of a fluid. The metal layer CM and the sensors C₁, C₂, . . . , C_{N} are housed in a chamber CC, and this chamber CC comprises an inlet NI and an outlet NO. The outlet NO is for example connected to an external pump (not shown) which supplies the chamber CC with a perfectly controlled flow of fluid between the inlet NI and the outlet NO.

The electronic nose NN further comprises computer means such as a microprocessor, an input/output communication stage, and means for connection and communication with other electronic devices, in particular with a computer processing circuit or a server. These means for connection and communication may be wired or wireless.

In the example presented here, the sensors C₁, C₂, . . . , C_{N} of the electronic nose NN are transducers sensitive to surface plasmon resonances (SPR) generated at the first face F1 of the metal layer CM in contact with the fluid in the chamber CC. By virtue of the principles of SPR measurement, a plasmon resonance generated at the first face F1, by polarization of the incident light, allows the sensors to measure variations in the refractive index of the fluid by detection of corresponding gray levels, by means of CCD cameras for example.

Local variations in the refractive index can thus be measured by the sensors C₁, C₂, . . . , C_{N} when different molecules present in the analyzed fluid are adsorbed, for example when volatile organic compounds present in a gas released by a item placed near the electronic nose NN are adsorbed by several of the sensors C₁, C₂, . . . , C_{N}. The adsorbed molecules are then imaged to determine the gray levels representative of their concentration.

For example, these sensors C₁, C₂, . . . , C_{N} are configured to adsorb various compounds such as heptanes, octanes, nonanes, ethanol, or beta-pinene. Each sensor of the electronic nose NN thus corresponds to a respective measured intensity I₁, I₂, . . . , I_{N} of the respective proportions of these compounds or fluids in this mixture. These proportions may optionally be normalized during the measurement or after it.

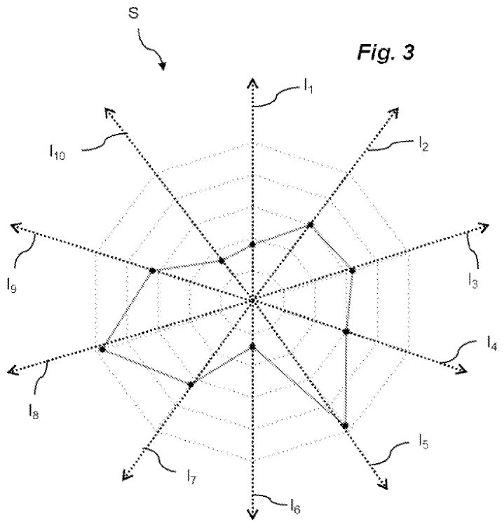

Measurement results obtained by 10 sensors C₁, C₂, . . . , C₁₀ of an electronic nose NN are represented in FIG. 3 in the form of a “radar” chart, these results forming an olfactory signature of a mixture of fluids originating from a given item P, for example from a perfume produced by a counterfeiter or from a banana just picked in Brazil.

In the context of measuring an olfactory signature of this item, this item releases a set of volatile organic compounds and each sensor adsorbs one or more of these compounds. The measurement of refractive index variations in the gas then allows identifying that different compounds react with different intensities according to each sensor.

In the example presented here, the intensities measured by the electronic nose NN are clearly separated and quantified according to each sensor, for example an intensity I₁ equal to 40 of an ethanol compound is measured by sensor C₁, an intensity I₂ equal to 60 of an octane compound is measured by sensor C₂, . . . and an intensity ho equal to 35 of a nonane compound is measured by sensor C₁₀. These intensities can also be represented by a vector with 10 components, equal to (40, 60, . . . , 35).

In the example presented here, the signal supplied by each sensor corresponds to the intensities measured and normalized relative to the set of sensors. In particular, the corresponding normalized intensity can be defined as the intensity measured by a given sensor divided by the norm, said norm being equal to the square root of the sum of the squares of the intensities measured by each sensor. This normalization makes it possible to separate out the intensity of the signature itself, which is advantageous for example when the concentration of fluid present in the mixture, or the odor, of the item is too strong, which results in saturation of the corresponding sensor. As the relative proportions are respected during normalization, it is possible to compare different signatures acquired over time by the same type of electronic nose. For each olfactory signature acquired, a normalization of the intensities corresponding to the respective proportions of the fluids then also makes it possible to estimate a model of olfactory signatures by means of multivariate analysis applied to normalized data.

Thus, a given intensity reflects the respective proportion of fluids adsorbed by the various sensors of the electronic nose NN, and therefore the concentration of these compounds in the mixture of fluids from the item. For 10 sensors we therefore obtain, for a given item, 10 spokes each corresponding to the response of a sensor. An olfactory signature is thus represented on the radar chart by a surface area whose shape varies depending on the odor released by the item at a given time.

We can therefore compare different surfaces in order to distinguish the olfactory signatures of a given item at different times, the differences between these surface areas or signatures possibly being the consequence of internal transformations of the item, for example due to forgery or maturation, or aging of the electronic nose NN due to degradation of its sensors.

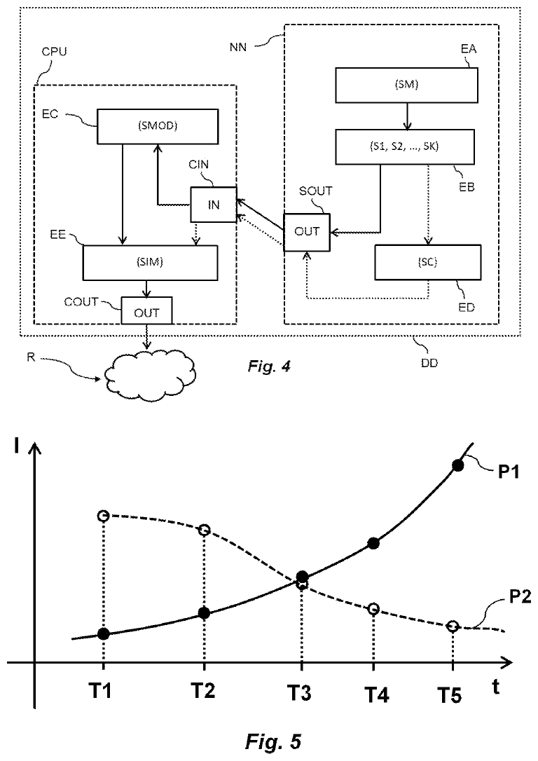

FIG. 4 illustrates a flowchart representing a method according to an exemplary implementation of the invention.

During a step EA, an electronic nose NN is used to acquire, as input, an olfactory signature SM of a mixture of fluids originating from a given item P. The signature is acquired by the set of sensors comprised in the electronic nose NN, for example 25 sensors C₁, C₂, . . . , C₂₅.

During a step EB, the step EA is repeated a first K times to acquire the same number of corresponding olfactory signatures. According to different variants, these K olfactory signatures can be acquired for the same given item P at times T1, T2, . . . , TK which are very close to each other in order to provide a more precise measurement by calculating means, which in particular allows calibrating an imperfect electronic nose NN. These K olfactory signatures can also be acquired from several items of the same type, for example several bananas of the same container, to provide a general measurement. These K olfactory signatures can also be acquired from the same perishable item at successive times T1, T2, . . . , TK which are distanced from each other, to provide olfactory signatures representative of successive maturation phases of this perishable item over time, or to define a model of evolution of its olfactory signature over time, as explained below.

In the example shown here, the K acquired signatures are stored in a memory, for example in the form of a first number K of pairs (S1, T1), (S2, T2), . . . , (SJ, TJ), . . . (SK, TK) where the Jth olfactory signature SJ is measured at a corresponding time TJ, J being a smaller number than the first number K. Alternatively, the K signatures are recorded in a memory in the form of a number K of triplets (S1, T1, P1), (S2, T1, P2), . . . , (SK, TY, PZ), where “Y” is the number of measurement times and “Z” the number of items, the sum of the numbers “Y” and “Z” being equal to the first number K.

According to different variants, the olfactory signatures of a single perishable item can thus be measured over the course of a supply chain, or the olfactory signatures of a plurality of perishable items that are part of the same lot, for example a dozen bananas of the same bunch stored in a transport container. In this case, a single olfactory signature can be used to represent all items in the lot, or to allow subsequent calibration of sensors of the electronic nose NN.

FIG. 5 shows a simplified example of olfactory signatures acquired successively over time at five times T₁, T₂, . . . , T₅, these olfactory signatures being defined by the intensity I as measured by a single sensor C₁. Represented is the case of a first perishable item P1 corresponding to the 5 black points, and a second perishable item P2 corresponding to the 5 white points. In this example, one can see that the proportion of fluid measured by this sensor in a mixture of fluids from P1 decreases over time while the proportion of the same fluid measured by the same sensor in a mixture of fluids from P2 generally decreases over time, resulting in two very different olfactory signatures.

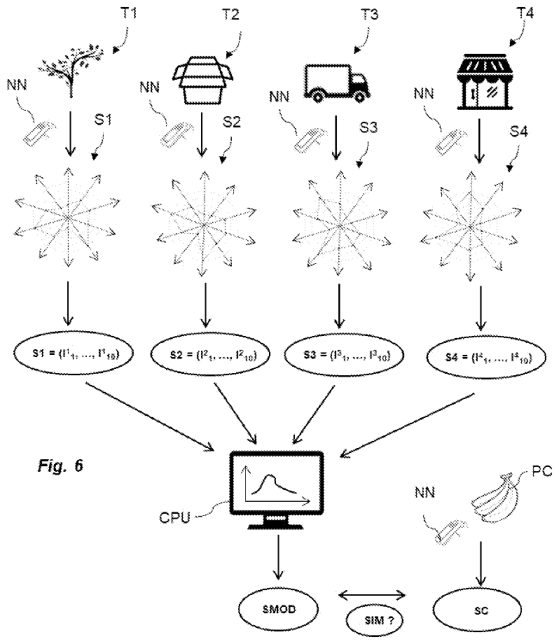

FIG. 6 shows a flowchart outlining an exemplary implementation of a method according to the invention in the context of a supply chain of items.

We consider here the case of a given perishable item P, in particular bananas of the same type, which is provided by a producer to a distributor over the same supply chain. It is assumed that these bananas are harvested during phase T1, packaged and stored during phase T2, transported by vehicle during phase T3, and distributed in stores during phase T4.

At any time during these four phases T1, T2, T3, and T4, an electronic nose NN can be used to acquire respective olfactory signatures S1, S2, S3 and S4 from banana samples representative of one of these phases.

Depending on the state of the bananas in each of these phases, different olfactory signatures are acquired over time. In the case where the electronic nose NN comprises 10 sensors, the olfactory signature S1 corresponds to 10 respective proportions I¹₁, . . . , I¹₁₀ of fluids in the mixture released by these bananas during phase T1. Depending on environmental conditions, supply conditions, and the proper maturation of these bananas over time, the olfactory signature S2 corresponding to the 10 proportions I²₁, . . . , I²₁₀ of the mixture released by the bananas during phase T2 is different, and so on for the 10 proportions measured by the sensors during phase T3 and for the 10 proportions measured by the sensors during phase T4. The representations of the four signatures S1, S2, S3 and S4 in the form of a “radar” chart are therefore different.

Each of these signatures is then transmitted to a computer processing circuit CPU, in order to estimate a model SMOD of the olfactory signature of the bananas for all of the phases T1, T2, T3 and T4, and taking into account its evolution at over time. This model SMOD is estimated using statistical analysis, as described below.

At any time, the electronic nose NN can be used to measure, and compare with the model SMOD, a current measurement SC of an olfactory signature of a current item PC, for example a sample from bananas dropped during an intermediate time T5 between the two phases T2 and T3. The result of this comparison makes it possible to identify and estimate a similarity SM between the current measurement SC and the model SMOD.

Returning to FIG. 4, steps EA and EB are implemented by an electronic nose NN. After step EA, following steps EA, EB or simultaneously with one of the two steps EA and EB, an electronic nose of the same type as NN, and preferably identical, is used to perform a current measurement SC of the olfactory signature of a current item PC, of the same type as the given item P. This makes it possible to subsequently compare the current measurement SC with one or more other olfactory signatures, and in particular with a model of the olfactory signature of the given item P established during another step.

The K olfactory signatures acquired during step EB are then transmitted to a computer processing circuit CPU which applies a statistical analysis to them in order to estimate a model SMOD of the olfactory signature of the given item P. The manner in which this model is estimated will be detailed in relation to the following figures.

In the example shown here, the electronic nose NN is connected to the computer processing circuit CPU and are both comprised in a device DD for identifying the given item P by its olfactory signature in accordance with this document. The processing circuit CPU is connected to the electronic nose and configured to implement the method according to one of the preceding claims.

After step EB, the K olfactory signatures are transmitted to an output module SOUT of the electronic nose NN which is in communication with an input module CIN of the computer processing circuit CPU, these two modules forming a communication interface between the electronic nose NN and the computer processing circuit CPU.

The device DD further comprises a communication module (not shown) for connecting said device to an external network R, and for exchanging data with other devices via said network. For example, the communication module can be a Wifi or Ethernet network interface, or a Bluetooth communication module. Preferably, the communication module also comprises a data reception module and a data transmission module.

Once the model SMOD has been established, it is compared with a current measurement SC of the olfactory signature of a current item PC, for example originating from a sample from bananas. The measurement SC is acquired during step ED. For example, the measurement SC is acquired in any of phases T1, T2, T3, T4, the SC pertaining to the same type of bananas as that used to establish the model SMOD. As with the K olfactory signatures, the measurement SC is transmitted to the output module SOUT of the electronic nose which communicates it to the input module CIN of the computer processing circuit CPU. The input module CIN then compares the measurement SC to the model SMOD in order to estimate a similarity SIM with the item PC. This similarity estimate SIM is then transmitted to an output module COUT of the computer processing circuit CPU, which communicates it to an external network R, for example a server or any other device enabling secure processing of the outgoing data, for example by means of processing result data from said similarity estimate, in particular to protect these data against falsification, for example via a circuit for data processing by blockchain (not shown).

In another example (not shown), the method is also applicable to the case of a non-perishable item P, for example sea salt collected during phase T1, transported during phases T2 and T3, and finally distributed during phase T4. In this case, the calibration, precision, and/or reliability of a same electronic nose NN can be verified at any time by comparing at least two olfactory signatures of a mixture of fluids from the item P. Precise control of the electronic nose NN can therefore be carried out by estimating a similarity SIM between a current measurement SC of the olfactory signature of a sample of salt from a current item PC during any one of phases T1, T2, T3 and T4 and the established model of the olfactory signature of the salt P, on the basis of the latter's olfactory signatures during all phases T1, T2, T3 and T4 or during some of them.

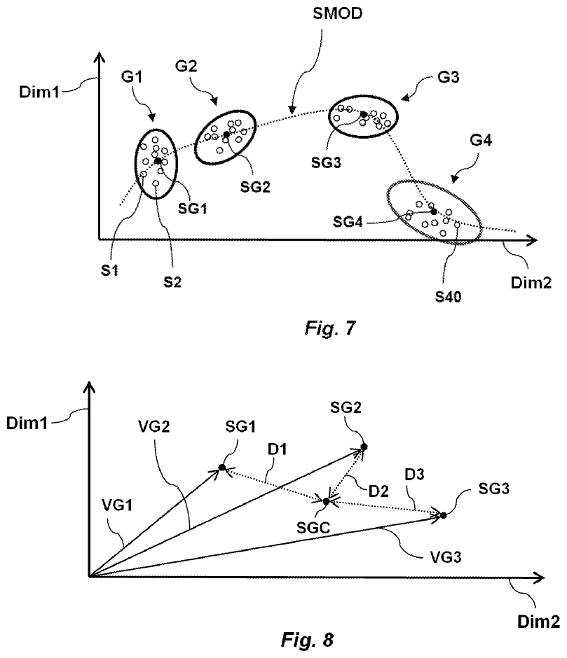

FIG. 7 shows an example of results obtained by applying a statistical analysis to 40 olfactory signatures S1, S2, . . . , S40 acquired from a same type of given item P.

In this case, these 40 olfactory signatures are acquired for the same perishable item, for example bananas originating in Brazil, during 4 successive maturation phases. According to one variant, these 40 olfactory signatures can also relate to those corresponding to 10 bananas taken from 4 different samples.

In the example presented here, an estimate of a model SMOD of the olfactory signature of the given item P is implemented via the application of multivariate analysis. Multivariate analysis consists of performing a dimensional reduction of the acquired data.

In particular, the multivariate analysis which is applied is principal component analysis, which makes it possible to determine a plurality, here a second number L, of parameters defining a respective state of the item in an L-dimensional space, L defining two dimensions Dim1 and Dim2 in FIGS. 7 and 8.

In this case, each olfactory signature represents the respective proportions of fluids from P as measured by 25 sensors C₁, C₂, . . . , C₂₅ of an electronic nose NN. Before applying multivariate analysis, the given item P is identified by 40*25 values of proportions, and can therefore be represented by 40 points in a 25-dimensional space. After applying multivariate analysis, the item P can be represented by 40 points, each comprising two coordinates, in a 2-dimensional space. In a non-limiting manner, the number L of dimensions is comprised between 1 and 10 and preferably between 1 and 5, and in all cases is less than the number of sensors.

Other analytical methods may be used. As non-limiting examples, mention may be made of principal component analysis, or PCA; multidimensional scaling, or MDS; principal component regression, or PCR; partial least squares regression, or PLS; partial least squares discriminant analysis, or PLS-DA. Most of these methods, in particular PLS-DA, have the advantage of incorporating prior learning from different groups of samples that have undergone similar processing, which optimizes the separation of points into different groups.

A multivariate analysis thus makes it possible to reduce the information corresponding to 25 measured proportions of an olfactory signature and to reduce them to 2 principal components. A limited number of principal components, ideally the most significant, are chosen to explain the variability of the olfactory signatures in an optimal manner.

In the example presented here, several olfactory signatures are therefore grouped into different groups after application of multivariate analysis. The 40 olfactory signatures of the perishable item form 4 clouds each comprising 10 points grouped into 4 groups G1, G2, G3, and G4 in this space of reduced dimensions. These clouds of points, or centroids, thus determine groups having in particular the shape of an interval (in 1 dimension), a circle (in 2 dimensions), a sphere (in 3 dimensions), or a hyper-sphere (in more than 3 dimensions). Multivariate analysis therefore makes it possible to group together the olfactory signatures represented in L-dimensional space, and to estimate variabilities with respect to the centroids, these variabilities possibly being due, for example, to maturation of the perishable item that we wish to identify or to aging of the sensors of the electronic nose over time.

If the number of dimensions is equal to 2, a virtual olfactory signature can be defined that represents the set of closest signatures in a given group, here in this case 4 centers SG1, SG2, SG3, and SG4, respectively corresponding to groups G1, G2, G3, and G4. These virtual olfactory signatures define a centroid or center of mass of the corresponding cloud of points (or, in the case of a single dimension, its median) as well as a corresponding radius (not shown). Such a radius is for example defined by the confidence interval of the reference points. The centroids can for example enclose 95% or 99% of the variability of each group, etc. A radius can also be defined by a distance calculated between the center and the furthest point, as detailed below.

In connection with FIGS. 7 and 8, and for each point corresponding to an olfactory signature of an item to be identified, it is possible to determine a distance D between several points and/or several phase vectors. A distance defined in an L-dimensional space can be of different types, for example an absolute value, a Euclidean distance, or a distance between a given number of nearest neighbors. Taking as the reference point the origin of the reference system of the L-dimensional vector space, a distance D is generally a norm “L”. In a 1-dimensional space, the distance can be the norm “1”, given by the sum of the absolute values. In a 2-dimensional space, the distance can be the square root of the sum of the squares. In an L-dimensional space, the norm L is the “1/Lth” root of the sum of the elements to the L^{th} power, and so on.

In both cases, these 40 signatures were acquired using the same electronic nose NN comprising 25 sensors, each sensor being sensitive to a different fluid likely to be present in a mixture of fluids obtained from P.

A model SMOD can then be estimated on the basis of interpolation of a path traveling through each group of points, and in particular through each of the centers of mass of these groups. Alternatively, the application of a regression to the principal components makes it possible to obtain an expected path, which may or may not be linear.

FIG. 8 shows an example of a comparison between a current item and given items using a set of phase vectors in a 2-dimensional space.

In the case of an L-dimensional space, defining a distance makes it possible to quantify and compare the maturation phases of a perishable item. An indicator phase vector in an L-dimensional space is typically defined by a set of coordinates X1, X2, . . . , XL in this space, said coordinates being used to calculate distances between the phase vectors.

In the present example, a first vector VG1 with two components designates the position of a first center of mass SG1, a second vector VG2 with two components designates the position of a second center of mass SG2, and a third vector VG3 with two components designates the position of a third center of mass VG3.

In this example, a point SGC corresponding to the current measurement SC is represented in 2-dimensional space Dim1 and Dim2 after application of multivariate analysis. We then determine which center of mass the point SGC is closest to by relative comparison of the distances separating this point from the various centers of mass, which allows estimating to which group and therefore which maturation phase the current measurement SC of the current item PC is closest. In practice, a virtual phase vector VGC (not shown) can be determined to correspond to point SGC. On the basis of the comparison of the distance D1 defined between phase vector VG1 and VGC, of the distance D2 defined between VG2 and VGC, of the distance D3 defined between VG3 and VGC, we can deduce that SGC is closest to SG2 in the space of dimensions Dim1 and Dim2. Indeed, as distance D2 is smaller than distances D1 and D3, we deduce from this comparison that the current item PC most probably belongs to group G2, and that this is probably the closest to the corresponding maturation phase.

In the example presented here, at least one of the distances determined between the current measurement represented in L-dimensional space and one of the centers of mass makes it possible to define a similarity SIM, also quantified, between the current item PC and the given item, by means of the estimation of the model SMOD.

Advantageously, the probability that a current item is in a given maturation phase can also be quantified by means of the distance separating the vector VGC from the phase vector associated with the center of mass of the group corresponding to this maturation phase.

In general, data resulting from an estimate of similarity SIM between the current item and the given item comprises data selected among any olfactory signature, any phase vector, any olfactory signature model, and/or any distance, established as explained above.

In the example presented here, these result data are processed in order to be protected against falsification. In particular, these result data are processed by blockchain.

For example, this processing is implemented by the computer processing unit CPU, which can be configured to encode the result data by placing them in a checksum (“hash”) of a blockchain. Alternatively, this processing is implemented by a circuit for processing data by blockchain CBC (not shown), which is in communication with the computer processing circuit CPU (for example a succession of communicating servers or the like). This communication can be continuous or irregular. Alternatively, the computer processing circuit CPU comprises this circuit CBC or itself constitutes this circuit for processing data by blockchain.

In particular, all of these data are included in one or more hashes defining an encoded data item, for example in hexadecimal hashes. In a non-limiting manner, these hashes can be hashes determined by algorithms of the MD5 or SHA type, which have the advantage of protecting the format of the included data such that any unauthorized read attempt automatically results in modification of this format, therefore directly identifiable. Advantageously, in addition, such signatures can be protected with an encryption cipher (RSA or other) such that decryption may require at least one secret key.

A blockchain using these hashes makes it possible to store data in a secure database, and/or to validate other data stored in the same database. This database may include one or more servers, in communication with the computer processing circuit CPU and possibly with one or more local or remote terminals, via a network such as the Internet.

During a first step of data processing in the example presented here, CBC can receive data from the electronic nose NN, from the computer processing circuit CPU, or from a secure database, and possibly may record them locally in a CBC memory. A CBC processor is configured to extract the data from the secure database.

In a next step, the CBC processor can generate metadata comprising blockchain data. These metadata include additional protections, in particular via the storage of a hash which makes it possible to encrypt each piece of data, this storage forming a block “A” of the blockchain. These metadata may contain information about other blocks in the blockchain and/or the value of a hash, for example a hash of another block. In a next step, and for each block “A”, the CBC processor generates a hash of the previous block “A−1”.

For example, to determine a hash of a block “A”, all data of this block “A” are concatenated with the value of the hash of the block “A−1” which precedes A, which provides a new hash, and so on. Gradually, a plurality of blocks comprising the encrypted data are thus generated. Since encryption of the data of a given block depends on hashes constructed from each preceding block, the security of the data is thus increased.

This improves the protection of data against falsification, starting with the acquisition of an olfactory signature of the item P by an electronic nose NN, a blockchain, and in particular a blockchain using hashes among those described above.

Advantageously, it is thus possible to record in a secure manner a data item resulting from an estimate of similarity SIM between the current item and the given item, and subsequently to compare it with a current measurement for authentication purposes.

1-15. (canceled)

16. A method implemented by a computer processing circuit, connected to an electronic nose, for identifying a given item by an olfactory signature of the given item, said method comprising:
making use of the electronic nose comprising a plurality of sensors for detecting the presence of fluids likely to be present in a mixture of fluids originating from the given item, in order to obtain an olfactory signature of said mixture, the olfactory signature comprising respective proportions of said fluids in the mixture;
repeating the use of the electronic nose a first number K of times in order to acquire K olfactory signatures;
making use of the computer processing circuit in order to estimate, on the basis of said K olfactory signatures, a model of the olfactory signature of the given item;
acquiring, with an electronic nose of the same type, a current measurement of the olfactory signature of a current item of the same type as said given item; and
comparing the current measurement to said model, in order to estimate a similarity between the current item and the given item.

17. The method according to claim 16, wherein, the given item being a perishable item, the use of the electronic nose to obtain the first number K of olfactory signatures over time is carried out successively over time in order to acquire a succession of K olfactory signatures over time.

18. The method according to claim 17, wherein, the perishable item having several successive maturation phases over time, the K olfactory signatures are representative of said successive phases of the perishable item.

19. The method according to claim 17, wherein the computer processing circuit is used to estimate, on the basis of said K successive olfactory signatures, a model of the evolution over time of the olfactory signature of the perishable item.

20. The method according to claim 19, wherein the perishable item having several successive maturation phases over time, the K olfactory signatures are representative of said successive phases of the perishable item; and the comparison of the current measurement to the model of the evolution over time gives an estimate of similarity between the current item and the perishable item at a given maturation phase of the perishable item.

21. The method according to claim 16, wherein the model of the olfactory signature of the given item is obtained by multivariate analysis of the K olfactory signatures, each determined by the respective proportions of said fluids in the mixture, the model being defined in an L-dimensional space, the multivariate analysis being selected among principal component analysis or multidimensional scaling analysis.

22. The method according to claim 17, wherein the perishable item having several successive maturation phases over time, the K olfactory signatures are representative of said successive phases of the perishable item; the comparison of the current measurement to the model of the evolution over time gives an estimate of similarity between the current item and the perishable item at a given maturation phase of the perishable item; and the model is defined by a set of phase vectors in L-dimensional space, each phase vector characterizing a maturation phase of the perishable item.

23. The method according to claim 22, wherein a distance is estimated, in the L-dimensional space, between a point representing the current measurement and each phase vector, the smallest of the estimated distances characterizing a current state of maturation of the perishable item.

24. The method according to claim 23, wherein the distance is an absolute value, a Euclidean distance, or a distance between the point representing the current measurement and several nearest-neighbor phase vectors.

25. The method according to claim 16, wherein the number of sensors comprised in the electronic nose is less than or equal to 100, and preferably equal to 25.

26. The method according to claim 16, further comprising:
grouping several olfactory signatures into at least two groups, each group being defined by a center of mass of the signatures in said group, the distance of each signature from said center of mass being less than or equal to a predetermined distance;
comparing the distances between the current measurement and each of said groups, in order to associate the current item with the group to which the current item is closest.

27. The method according to claim 16, further comprising a processing of result data from said estimate of similarity between the current item and the given item, in order to protect said data against falsification.

28. The method according to claim 27, wherein said result data are processed by a blockchain.

29. A device for identifying a given item by its olfactory signature, the device comprising:
an electronic nose, comprising a plurality of sensors for detecting the presence of fluids likely to be present in a mixture of fluids originating from the given item,
a computer processing circuit, connected to the electronic nose in order to obtain an olfactory signature of said mixture, the olfactory signature comprising respective proportions of said fluids in the mixture,
and the computer processing circuit being configured to implement the method according to claim 16.

30. A computer program comprising instructions for implementing the method according to claim 16, when said instructions are executed by a processor of a processing circuit.
